(19) United States
(12) Patent Application Publication
(10) Pub. No.: US 2015/0360021 A1
(43) Pub. Date: Dec. 17, 2015

(54) MULTIPURPOSE MEDICAL INSTRUMENT CAPPING DEVICE

(71) Applicants: Maria Limdico, Orlando, FL (US); Shaun Masterson, Orlando, FL (US)

(72) Inventors: Maria Limdico, Orlando, FL (US); Shaun Masterson, Orlando, FL (US)

(21) Appl. No.: 14/301,772

(22) Filed: Jun. 11, 2014

(51) Int. Cl. A61M 39/20; A61M 39/12; A61M 5/178

(52) U.S. Cl. CPC A61M 39/20; A61M 5/178; A61M 39/12; A61M 2039/0205

(57) ABSTRACT

A multipurpose medical instrument capping device is disclosed. The device includes a syringe cap having a first end that is adapted to engage an access port of an IV line. The syringe cap also includes a second end that is adapted to engage a needleless opening on an IV flush syringe. The luer cap includes a closed end and an open end that is adapted to engage an IV luer. The syringe cap and the luer cap are removably secured together by a coupler.

### TECHNICAL FIELD

The present invention relates generally to the field of medical instruments, and more particularly to a multipurpose capping device for medical instrument access ports that can eliminate waste and prevent the spread of infection.

### BACKGROUND

The statements in this section merely provide background information related to the present disclosure and may not constitute prior art.

Within the medical field, and in particular the area of infusion of fluids to or from a patient, there is a need to prevent the transmission of pathogens such as bacteria and/or viruses into a patient from a potentially contaminated surface of a medical instrument. The transmission of pathogens into a patient may result in an infection that could be life threatening. Specific to healthcare settings, the term “nosocomial infection” describes those infections that originate from or occur in a hospital or such setting. In the United States alone, hospital-acquired infections are estimated to occur in at least 5% of all acute care hospitalizations. The estimated incidence is more than two million cases per year, resulting in an added expenditure in excess of $4.5 billion. In addition to the above, it is believed that such hospital-acquired infections are responsible for about 90,000 deaths a year.

Among all of the medical instruments employed within the health care industry, the devices believed to be most commonly responsible for the spread of life threatening hospital-acquired infections include: luer ports, vials, needle free valves, injection ports of a vessel, tubing, and catheters. With specific regard to IV sets, Infection Control Practitioners (ICP's) often cite failure by the caretaker to properly clean and/or cap access ports as a major source of these infections. One of the most common reasons cited by healthcare workers for failing to cap access ports is that many times, they do not have appropriate caps available, as these items are manufactured, shipped, sold and packaged separately from the other IV components.

In this regard, background FIG. 1 illustrates the components required to perform a simple IV flush. The components include an IV flush syringe 1 having a plunger for dispensing a saline solution through a needleless opening. IV flush syringes are manufactured with a disposable cap 1a. Once the IV set and the patient's IV line have been separated, it is necessary to ensure both ends are capped. As such, a sterile cap 2a must be procured in order to protect the luer of the IV set 2, and a separate access port cap 3a must also be procured in order to protect the IV line of the patient 5. The use and operation of the IV set, the flush syringe and the IV line are known to those of skill in the art, and in each instance, if/when the IV tubes are reconnected each of the caps are discarded.

As such, the current practice of utilizing several different shaped and sized caps to protect a patient against infection causes a burden on healthcare workers who often forget, or are unable to acquire one of the caps.

Accordingly, it would be beneficial to provide a multipurpose medical instrument capping device that can alleviate the above noted issues.

### SUMMARY OF THE INVENTION

The present invention is directed to a multipurpose medical instrument capping device. One embodiment of the present invention can include a syringe cap that is secured to a luer cap via a coupler. The syringe cap can include a first end for engaging an access port on a patient's IV line. The syringe cap can also include a second end for engaging a needleless opening on an IV flush syringe. The luer cap can include a closed end and an open end for engaging an IV luer.

In another embodiment, the interior portion of the luer cap can be sterilized, and the interior portion of the syringe cap can include a cleaning material.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of an IV set, an IV line and an IV flush syringe in accordance with background art.

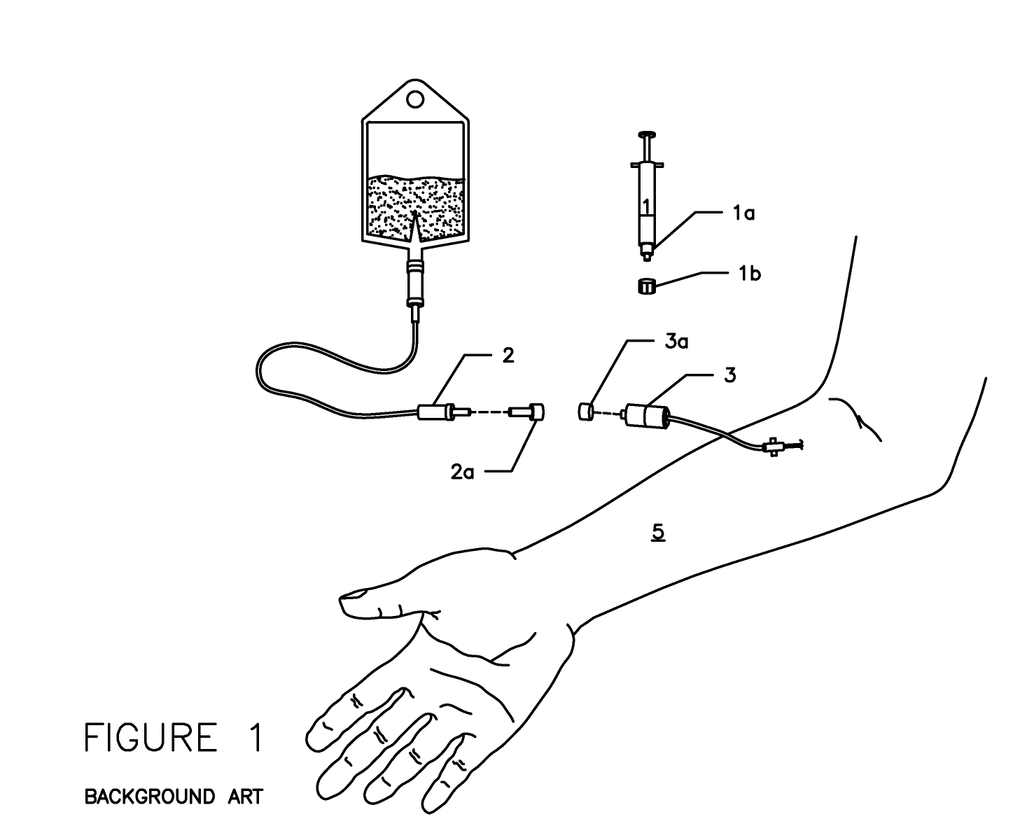

FIG. 2A is a side view of a multipurpose medical instrument capping device in a closed orientation, in accordance with one embodiment of the invention.

FIG. 2B is a side view of a multipurpose medical instrument capping device in an open orientation, in accordance with one embodiment of the invention.

FIG. 3A is a perspective view of the syringe cap of the multipurpose medical instrument capping device, in accordance with one embodiment of the invention.

FIG. 3B is a side view of the syringe cap of the multipurpose medical instrument capping device, in accordance with one embodiment of the invention.

FIG. 4A is a perspective view of the luer cap of the multipurpose medical instrument capping device, in accordance with one embodiment of the invention.

FIG. 4B is another perspective view of the luer cap of the multipurpose medical instrument capping device, in accordance with one embodiment of the invention.

FIG. 5 is a front view of the coupler of the multipurpose medical instrument capping device, in accordance with one embodiment of the invention.

FIG. 6 is a side view of the multipurpose medical instrument capping device in operation, and in accordance with one embodiment of the invention.

FIG. 7 is a perspective view of the multipurpose medical instrument capping device in operation, and in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

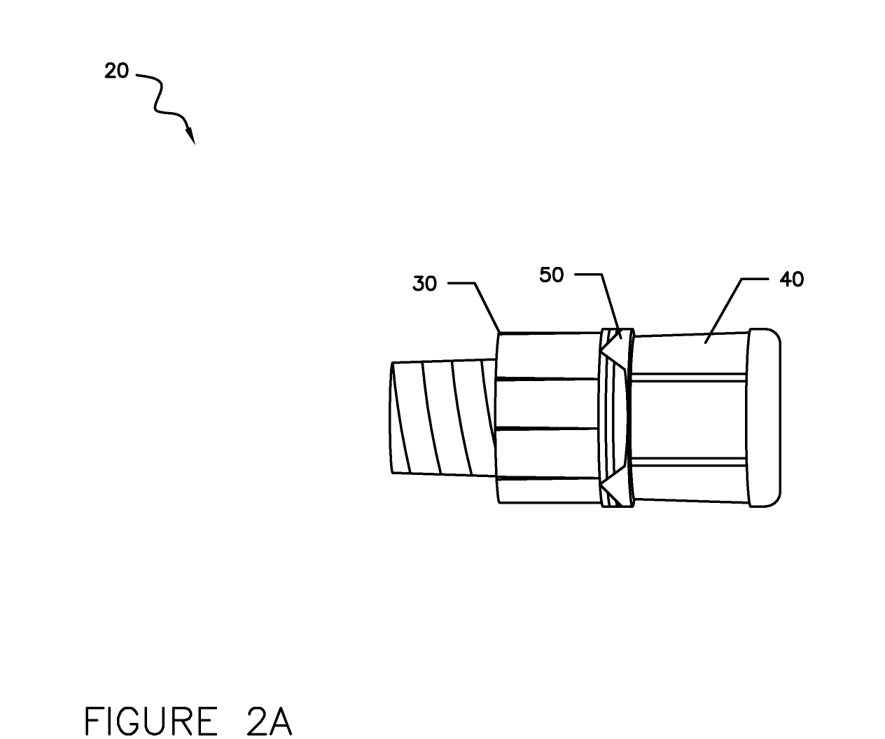

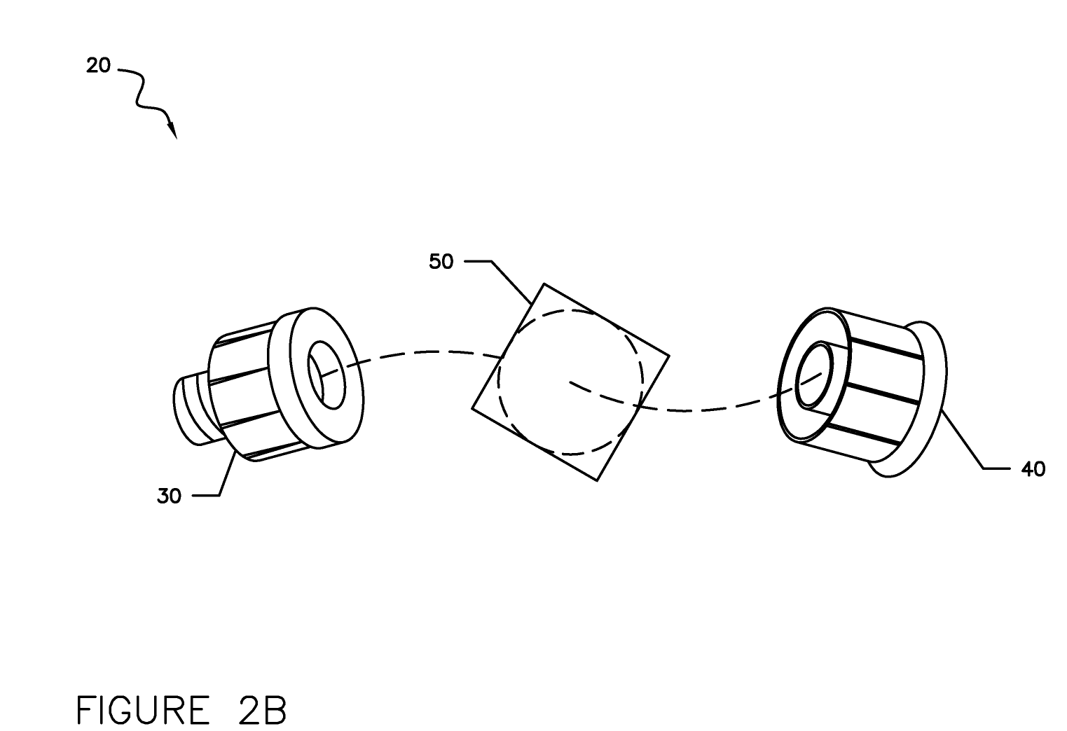

Identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 2B.

As described herein, the term “removably secured,” and derivatives thereof shall be used to describe a situation wherein two or more objects are joined together in a non-permanent manner so as to allow the same objects to be repeatedly joined and separated. Likewise, as described herein, the term “medical instrument,” and derivatives thereof shall be used to describe any tool or object that can be used in a medical setting and that can connect with the multipurpose cap described below. Several non-limiting examples of medical instruments include, but are not limited to, access ports on tubing sets (extension sets, T-connectors and IV sets), access ports on catheters (both peripheral and central lines), needle free valves, stopcocks, luer connectors, and other such components which must be capped to prevent pathogens from being delivered to a patient.

As each of these medical instruments are commercially available in standard sizes, the multipurpose cap can be constructed with fittings (e.g., threaded elements) to accommodate and engage the standard sized medical instruments. As will be known to those of skill in the art, threaded elements typically include lands and grooves for engaging objects having complementary threaded elements together via a twisting motion. Although described throughout this document as utilizing threaded elements, any number of other known fittings capable of creating a removable seal between two items are also contemplated. Accordingly, the device is not to be construed as limited to the use of threaded elements.

FIGS. 2A-5 illustrate one embodiment of a multipurpose medical instrument capping device 20 that is useful for understanding the inventive concepts disclosed herein. Although described below in conjunction with specific medical instruments, this is for illustrative purposes only, as the device 20 can be sized to accommodate a wide range of different medical instruments and is not to be construed as limiting to the uses described below.

FIGS. 2A and 2B illustrate the device 20 in an assembled and disassembled orientation, respectively. As shown, the device 20 can include an IV syringe cap 30 that is removably secured to a sterile luer cap 40 via a coupler 50.

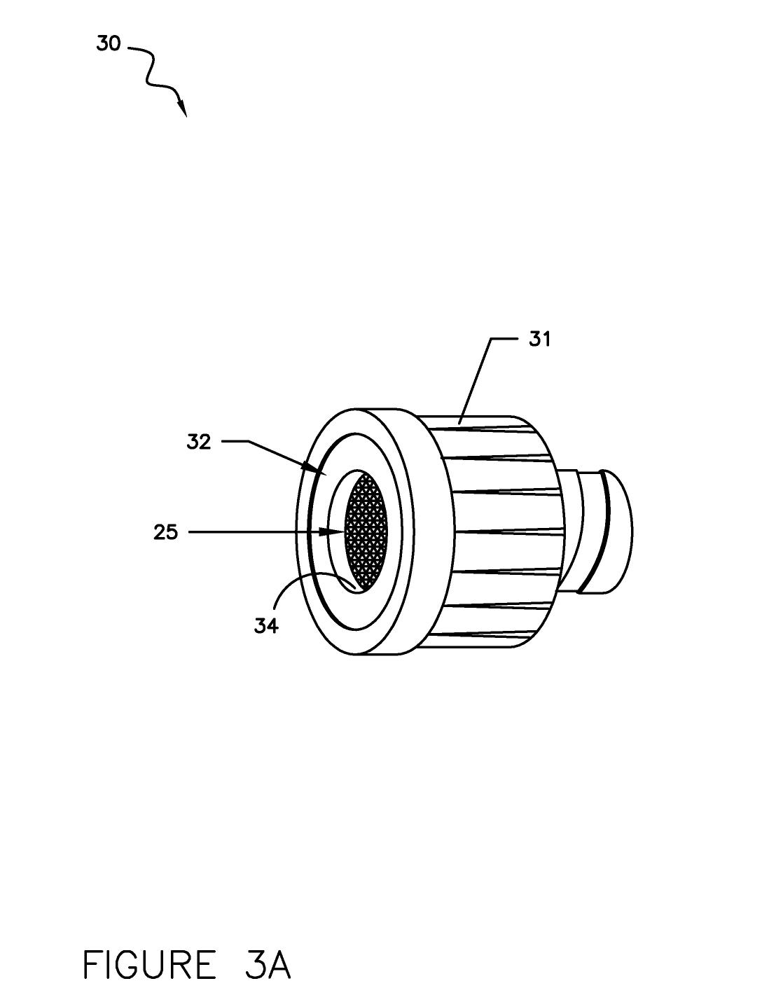

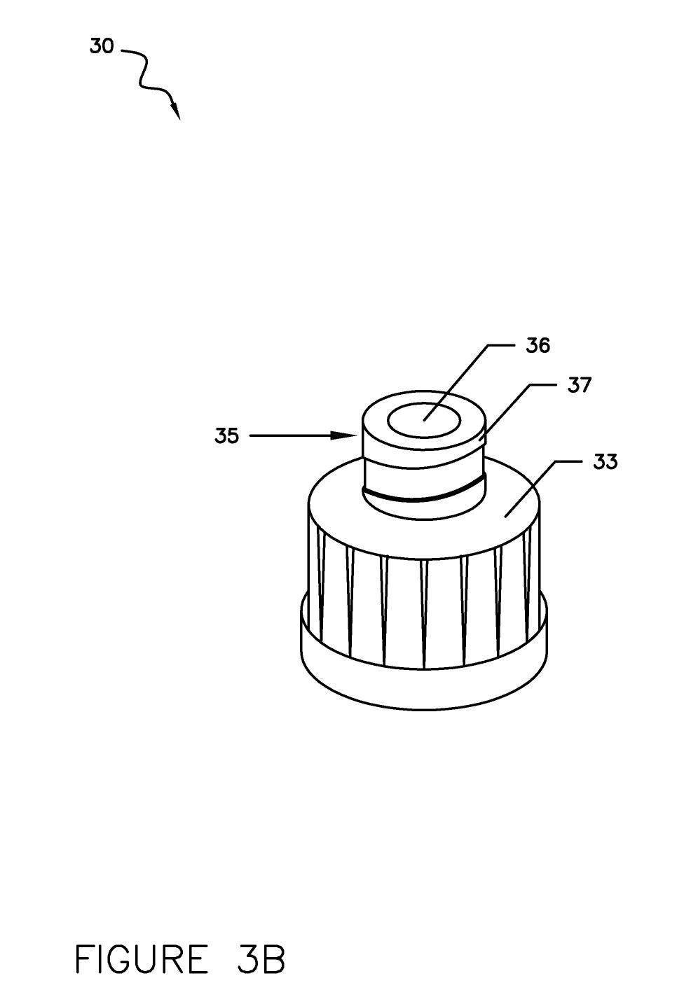

FIGS. 3A and 3B illustrate one embodiment of the IV syringe cap 30 that includes a main body 31 having an opening 32 at one end, a closed portion 33 at another end, and a generally hollow interior space. A plurality of threaded elements 34 can be disposed within the interior space of the main body and can function to engage the complementary threaded elements that are located on the access port of a patient's IV line. The cap 30 can also include an elongated generally hollow channel 35 having a channel opening 36 that extends linearly from the closed portion 33. The channel also including a plurality of threaded elements 37 that can be disposed along the outer surface of the channel, in order to engage complementary threaded elements on a flush syringe.

In one optional embodiment, a cleaning material 25 can be disposed within the interior space of the main body 31, and accessible via the opening 32. The cleaning material can function to thoroughly clean a patient's IV line while preventing the same from being contaminated. In the preferred embodiment, the cleaning material 25 can include an absorbent member such as cotton or foam, for example, that is soaked in a cleaning solution such as alcohol, for example. One suitable example of a cleaning material is described in U.S. Pat. No. 7,780,794, the entire contents of which are incorporated herein by reference.

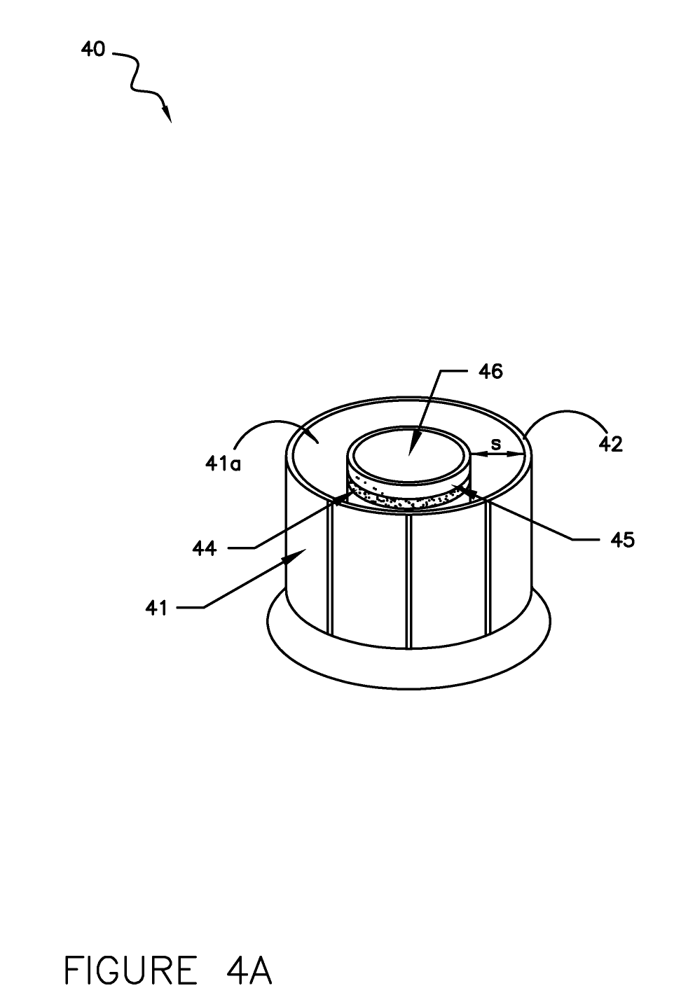

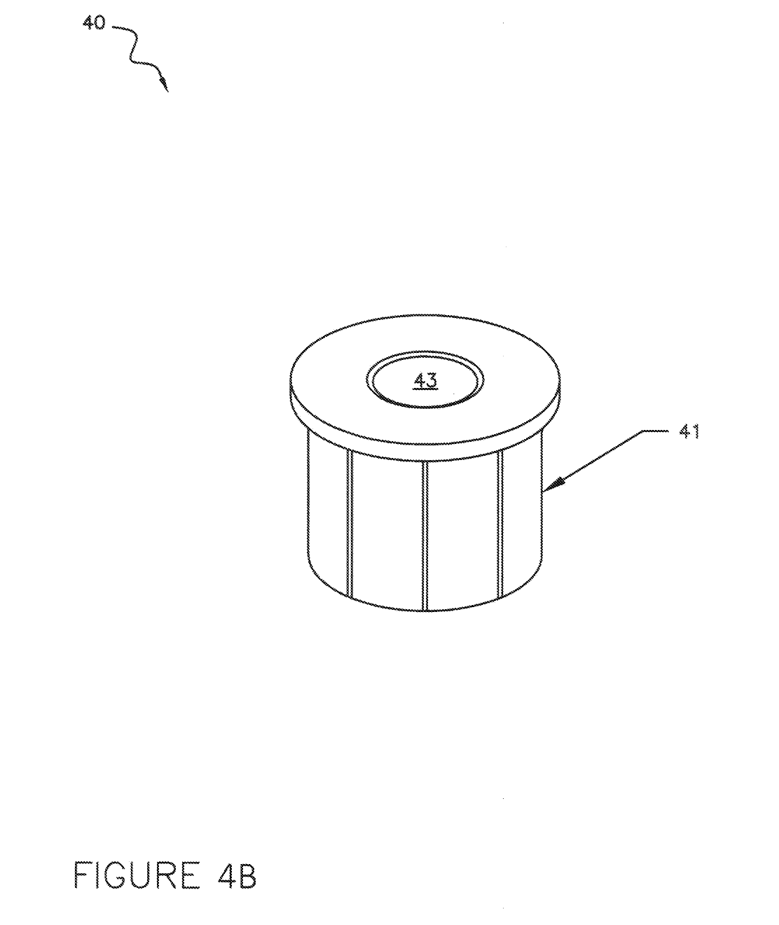

FIGS. 4A and 4B illustrate one embodiment of the luer cap 40. As shown, the cap can include a main body 41 having a main body opening 42 at one end, a closed second end 43, and a generally hollow interior space. A generally hollow channel 45 can be disposed within the interior portion of the main body and can extend from the closed second end 43 to the opening 42. The channel 45 can include a channel opening 46 along one end, and can preferably be centered within the main body so as to ensure a uniform space S between the channel 45 and the inside surface of the main body 41a. A plurality of threaded elements 44 can be disposed along the outside surface of the hollow channel 45 and can function to engage the complementary threaded elements located on an IV luer.

In the preferred embodiment, the inside surface of the main body 41a, and the entire interior channel 45 can be sterilized in accordance with known techniques, so as to prevent contaminating the IV luer and the sterile contents of the IV set. Moreover, both the inside surface of the main body 41a, and the entire interior channel 45 can be impregnated with alcohol or another such cleaning solution.

In the preferred embodiment, both the IV syringe cap 30 and the luer cap 40, including all subcomponents, can each be formed of a unitary piece of durable material such as polyethylene plastic, for example which is suitable and FDA approved for use in a medical environment. Of course, any number of other materials and construction methodologies are also contemplated.

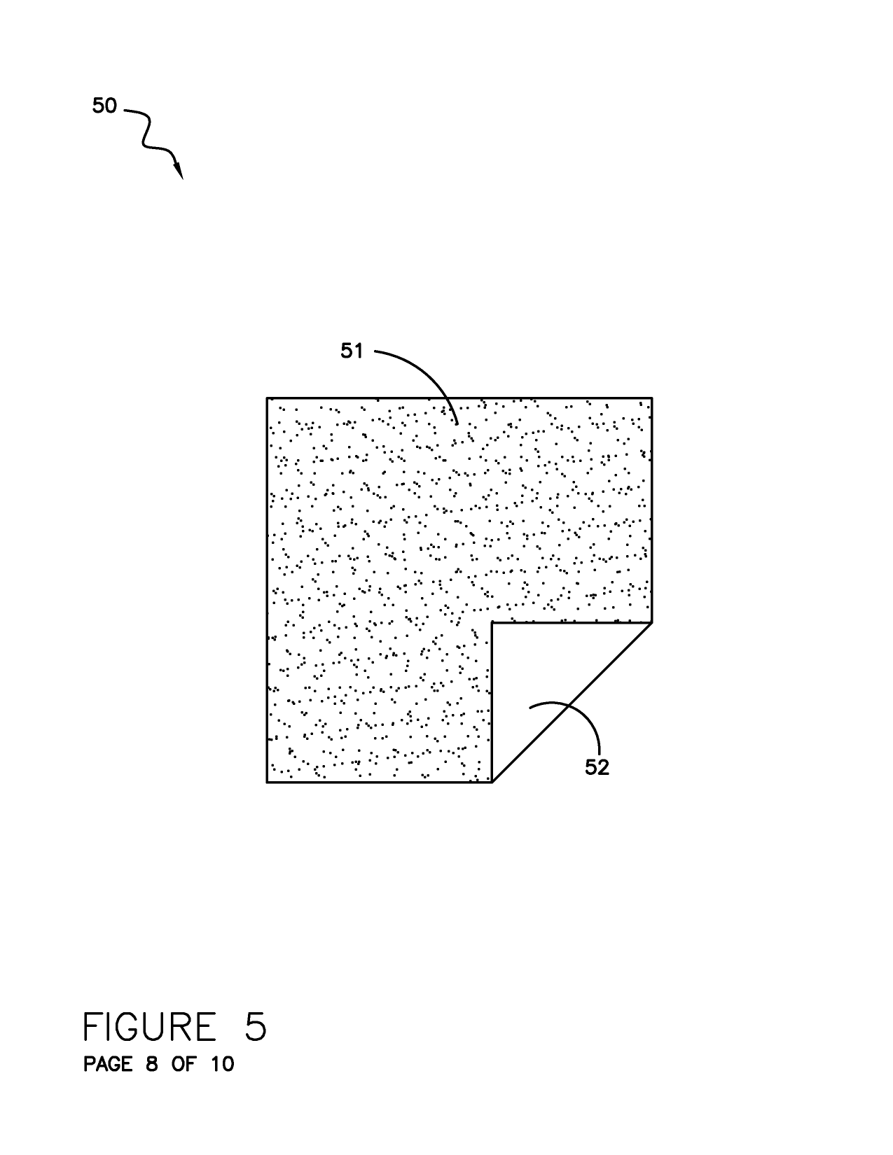

FIG. 5 illustrates one embodiment of a coupler 50 which can function to mate the IV syringe cap 30 and the luer cap 40 together in a removable manner. As shown, the coupler 50 can include a main body having a first surface 51 with a dimension that is suitable for covering the entire syringe cap opening 32, and a second surface 52 with a dimension that is suitable for covering the entire luer cap opening 42.

As noted above, it is preferred that the interior surface of the luer cap 40 be sterile, therefore in the preferred embodiment, the coupler 50 can be constructed from a nonporous and waterproof material such as foil for example that is adhered to each of the caps via glue, solvent or thermal bonding, for example. Such a feature can function to ensure that the luer cap remains sterile, and to ensure that any cleaning material 25 located within the syringe cap also remains unspoiled.

Although illustrated as including a generally planar rectangular member, this is for illustrative purposes only, as the coupler can include any number of different shapes, sizes and thicknesses in order to securely mate with each of the IV syringe cap 30 and the luer cap 40 in a non-permanent manner wherein the two caps can be separated via a twisting or pulling motion by an end user.

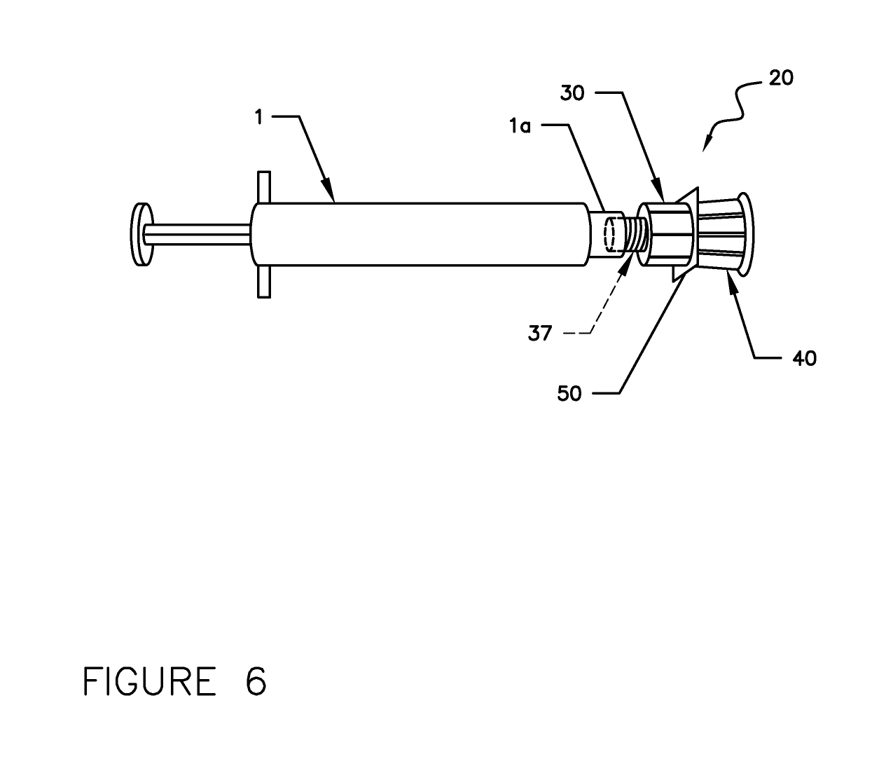

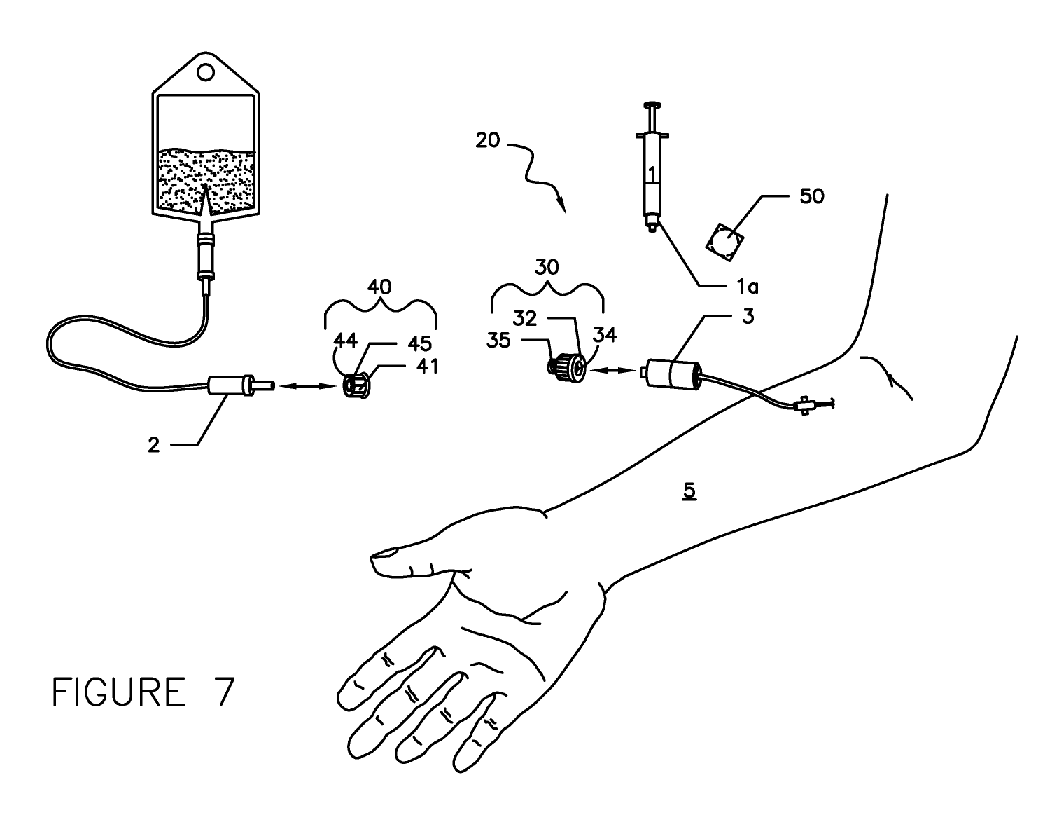

Now that a basic understanding of the inventive concepts has been disclosed, a method of using the same will now be described with respect to FIGS. 6 and 7.

Although not essential, in the preferred embodiment, the multipurpose capping device 20 can be pre-packaged with a conventional flush syringe 1, in order to ensure that each cap necessary to perform a flush procedure will be available to a caregiver. As such, in one embodiment, the threaded elements 37 located on the on the cap channel 35 can engage the complementary threaded elements 1a located within the needleless syringe opening.

Next, the device 20 can be separated as described above, and as shown in FIG. 2B. Once separated, the open end 42 of the luer cap 40 can be secured to the IV luer 2 via the threaded elements 44. At this time, the sterile internal channel 45 can make direct contact with the opening within the IV luer in order to prevent any contaminants from entering the sterile environment of the IV set.

Finally, after the patient's IV has been flushed with the flush syringe 1, the open end 32 of the syringe cap 30 can be secured to the access port 3 of the patient's IV via the threaded elements 34. Moreover, in the instances where a cleaning material 25 is located within the main body of the syringe cap, the cleaning material 25 can function to keep the access port clean at all times, thereby further reducing the risk of exposing the patient to infection.

As described herein, one or more elements of the multipurpose medical instrument capping device 20 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more of the above noted individual elements may be formed together as one continuous element, either through manufacturing processes, such as welding, casting, or molding, or through the use of a singular piece of material milled or machined with the aforementioned components forming identifiable sections thereof.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A multipurpose medical instrument capping device, said device comprising:
a syringe cap that includes
a main body having an opening disposed at a first end, a closed second end and a hollow interior space, said interior space including a plurality of threaded elements that are located adjacent to the syringe cap main body opening,
an elongated generally hollow channel extending outward from the closed end of the main body, said channel including a plurality of threaded channel elements that are disposed along an outside surface thereof;
a luer cap that includes
a main body having an opening disposed at a first end, a closed second end and a hollow interior space,
an elongated generally hollow channel is disposed within the hollow interior space and extending from the closed second end to the opening, said channel including a plurality of threaded elements that are located along an outside facing surface thereof; and
a coupler that is configured to removably secure each of the syringe cap and the luer cap together.

2. The device of claim 1, wherein the coupler further includes:
a main body having a first surface with a dimension that is greater than a dimension of the syringe cap main body opening; and
a second surface that includes a dimension that is greater than a dimension of the luer cap main body opening.

3. The device of claim 2, wherein the coupler main body is constructed from a nonporous waterproof material.

4. The device of claim 3, wherein the coupler is removably secured to each of the syringe cap and the luer cap via, at least one of glue, a solvent and thermal bonding.

5. The device of claim 1, further comprising:
a cleaning material that is disposed within the hollow interior space of the syringe cap.

6. The device of 5, wherein said cleaning material consists of:
an absorbent member that is soaked in a cleaning solution.

7. The device of claim 1, wherein an entirety of the interior space of the luer cap main body is sterile.

8. The device of claim 7, wherein said coupler is constructed from a material that functions to secure the syringe cap and the luer cap together and maintain the sterile interior space of the luer cap.

9. The device of claim 1, wherein the plurality of threaded elements of the syringe cap include a dimension that are configured to engage an IV access port, the plurality of threaded channel elements include a dimension that are configured to engage a needleless syringe opening, and the plurality of threaded elements of the luer cap include a dimension that are configured to engage an IV luer.

10. The device of claim 1, wherein the syringe cap and the luer cap are each formed from a unitary piece of FDA approved plastic.

11. An IV flush syringe, comprising:
a main syringe body having a plunger at one end, and a needleless opening disposed at an opposite end; and
a multipurpose medical instrument capping device comprising
a syringe cap that includes
a main body having an opening disposed at a first end, a closed second end and a hollow interior space, said interior space including a plurality of threaded elements that are located adjacent to the syringe cap main body opening,
an elongated generally hollow channel extending outward from the closed end of the main body, said channel including a plurality of threaded channel elements that are disposed along an outside surface thereof, said elements being in communication with a plurality of complementary threaded elements located along the needleless opening of the syringe,
a luer cap that includes
a main body having an opening disposed at a first end, a closed second end and a hollow interior space, said interior space including a plurality of threaded elements that are located adjacent to the luer cap main body opening,
an elongated generally hollow channel that is disposed within the hollow interior space and extending from the closed second end to the opening, and
a coupler that is removably secured to each of the syringe cap and the luer cap.

12. The syringe of claim 11, wherein the coupler further includes:
a main body having a first surface with a dimension that is greater than a dimension of the syringe cap main body opening; and
a second surface that includes a dimension that is greater than a dimension of the luer cap main body opening.

13. The syringe of claim 12, wherein the coupler main body is constructed from a nonporous waterproof material.

14. The syringe of claim 13, wherein the coupler is removably secured to each of the syringe cap and the luer cap via, at least one of glue, a solvent and thermal bonding.

15. The syringe of claim 11, further comprising:
a cleaning material that is disposed within the hollow interior space of the syringe cap.

16. The syringe of claim 15, wherein said cleaning material consists of:
an absorbent member that is soaked in a cleaning solution.

17. The syringe of claim 11, wherein an entirety of the interior space of the luer cap main body is sterile.

18. The syringe of claim 17, wherein said coupler is constructed from a material that functions to secure the syringe cap and the luer cap together and maintain the sterile interior space of the luer cap.

19. The syringe of claim 11, wherein the plurality of threaded elements of the syringe cap include a dimension that are configured to engage an IV access port, the plurality of threaded channel elements include a dimension that are configured to engage a needleless syringe opening, and the plurality of threaded elements of the luer cap include a dimension that are configured to engage an IV luer.

20. The syringe of claim 11, wherein the syringe cap and the luer cap are each formed from a unitary piece of FDA approved plastic.
